(19) United States
(12) Patent Application Publication
(10) Pub. No.: US 2010/0112543 A1
(43) Pub. Date: May 6, 2010

(54) PROCESSING SOFT TISSUE, METHODS AND COMPOSITIONS RELATED THERETO

(76) Inventors: Manh-Dan NGO, Rochester, NY (US); Arthur Gertzman, Flemington, NJ (US); Michael Kawas, Freehold, NJ (US); Katherine G. Truncale, Hillborough, NJ (US); Moon Hae Sunwoo, Old Tappan, NJ (US); Alison Lee, Piscataway, NJ (US); Richard Seeman, South Bound Brook, NJ (US); Carl Alexander DePaula, Malvern, PA (US); Jeffrey S. Cartmell, Freehold, NJ (US); David J. Gocke, Princeton, NJ (US); Carina Syring, Biel-Benken (CH); Rudiger von Versen, Wanditz (DE)

Correspondence Address: Sandra Kuzmich, Ph.D., Esq.;c/o FROMMER LAWRENCE & HAUG LLP, 745 Fifth Avenue, New York, NY, 10151, US

(21) Appl. No.: 12/534,613

(22) Filed: Aug. 3, 2009

Related U.S. Application Data
(63) Continuation-in-part of application No. PCT/US2008/052882, filed on Feb. 4, 2008.
Application No. PCT/US2008/052882 is a continuation-in-part of application No. PCT/US2008/052884, filed on Feb. 4, 2008.
Application No. PCT/US2008/052884 is a continuation-in-part of application No. PCT/US2008/052885, filed on Feb. 4, 2008.
Application No. PCT/US2008/052885 is a continuation-in-part of application No. 11/375,026, filed on Mar. 15, 2006.
(60) Provisional application No. 60/899,021, filed on Feb. 2, 2007.
(60) Provisional application No. 60/899,020, filed on Feb. 2, 2007.
(60) Provisional application No. 60/899,018, filed on Feb. 2, 2007.
(60) Provisional application No. 60/924,249, filed on May 4, 2007.
(60) Provisional application No. 60/662,078, filed on Mar. 16, 2005.

(51) Int. Cl. A01N 1/02; C12N 5/071; A01N 1/00

(52) U.S. Cl. 435/1.3; 435/325; 435/1.1

(57) ABSTRACT

In certain embodiments, the present invention relates to a process for preparing skin removed from a human donor, including a living human donor, and removing cellular components and forming a decellular matrix having as major components collagens and elastins while disinfecting the tissue. In other embodiments, the present invention relates to a process for treating a decellularized soft tissue by freezing the same at a plurality of decreasing temperatures at atmosphere or higher such that there is formation of ice crystals having a size greater than 2.0 and lyophilizing the soft tissue under vacuum to remove the water to less than 6% forming a porous matrix.

### RELATED APPLICATIONS

This application is a continuation-in-part of International Application Nos. PCT/US2008/052882, PCT/US2008/052884, and PCT/US2008/052885, each filed Feb. 4, 2008 and claiming the benefit of priority of U.S. Provisional Application Ser. Nos. 60/899,021, filed Feb. 2, 2007; 60/899,020, filed Feb. 2, 2007; 60/899,018, filed Feb. 2, 2007; and 60/924,249, filed May 4, 2007. This application is also a continuation-in-part of U.S. Ser. No. 11/375,026, filed Mar. 15, 2006, which claims the benefit of priority of U.S. Provisional Application Ser. No. 60/662,078, filed Mar. 16, 2005. The specifications of the foregoing applications are hereby incorporated by reference in their entirety.

### FIELD OF INVENTION

The present invention is generally directed toward methods of treatment of allograft soft tissue taken from, for example, a human donor, including hair removal, decellularizing and disinfection for implantation into another human being as well as methods of treatment of allograft soft tissue to increase porosity of the tissue.

### BACKGROUND OF THE INVENTION

Techniques for restoring structure and function to damaged tissue are used routinely in the area of reconstructive surgery. Tissue transplantation is another way of restoring function by replacing or rebuilding the damaged tissue. However, problems exist when there is a transfer of biological material from one individual to another. Tissue rejection is a significant risk associated with transplantation, even with a good histocompatability match. Immunosuppressive drugs such as cyclosporin and FK506 are usually given to the patient to prevent rejection. These immunosuppressive drugs however, have a narrow therapeutic window between adequate immunosuppression and toxicity. Prolonged immunosuppression can weaken the immune system, which can lead to a threat of infection. Also, tissue when lyophilized becomes hard and is difficult to rehydrate so that it is flexible for surgical use. In an attempt to solve this problem, physical puncturing and slitting of the implant has been utilized, which can reduce the implant's structural integrity and introduce contamination into the implant.

The advantages of retaining an acellular matrix, composed primarily of a collagenous component, have been explored by Klaus and Duhamel (WO 84/0488)) for the production of sterile body implants. In this method, a variety of tissues were extracted sequentially with non-ionic and ionic detergents to yield structures essentially free of cellular membranes, nucleic acids, lipids and cytoplasmic components. The treatment consists of sequential extractions with a non-denaturing detergent and a denaturing detergent to form an acellular matrix of collagen.

U.S. Pat. No. 4,776,853 issued Oct. 11, 1988 pertains to a process for preparing biological material for implant in a mammal's cardiovascular system, respiratory system or soft tissue. The process comprises: (1) isolating a desired tissue sample of the biological material from a donor; (2) extracting the tissue sample with an hypotonic buffer solution at a mild alkaline pH, the buffer solution including active amounts of proteolytic inhibitors and antibiotics; (3) extracting the tissue sample with a buffered solution having a high concentration of salt, the solution being at a mild alkaline pH and including a non-ionic detergent with protease inhibitors and antibiotics; (4) subjecting tissue sample to enzymatic digestion in a buffered saline solution, the enzymes consisting of purified protease-free dioxyribonuclease and ribonuclease; (5) extracting the tissue sample with an anionic detergent at a mild alkaline pH; and (6) storing the tissue sample in physiologic buffered solutions.

Another soft tissue process is shown in U.S. Pat. No. 6,734,018 issued May 11, 2004 which relates to a process for preparing an acellular soft tissue graft for implantation into a mammalian system. The process extracts a soft tissue sample with an extracting solution including one or more nonionic detergents and one or more endonucleases, to produce extracted tissue and treats the extracted tissue with a treating solution including one or more anionic detergents, to produce a treated tissue. The treated tissue is washed with a decontaminating solution including one or more decontaminating agents to produce the acellular soft tissue graft; and acellular soft tissue graft is then stored in a storage solution comprising one or more decontaminating agents.

The soft tissue process of the '018 patent includes the steps of: isolating from a suitable donor a desired tissue sample of the biological material; extracting the tissue with mildly alkaline hypotonic buffered solution of an endonuclease such as Benzonase® and a nonionic detergent formulation such as Allowash Solution™ optionally treating the tissue with a hypertonic buffered salt solution; extracting and treating the tissue with a mildly alkaline hypotonic buffered solution of sodium dodecylsulfate, optionally with 0.1 to 0.5 M sodium chloride rendering the solution hypertonic; washing the tissue with ultrapure water followed by a water solution of chlorine dioxide; and storage in a sealed container in isotonic saline, chlorine dioxide or 70% isopropanol.

A number of patents are directed towards punching holes in soft tissue implants or cutting slits in soft tissue implants. U.S. Pat. No. 6,197,036 pertains to a natural or synthetic material having a plurality of apertures cut therein. The natural material can be fibrous tissue, fascia including the fascia, dura, pericardium, striated muscle or part of the vaginal wall. Tissue from allogenic sources may be freeze dried to produce the immune response to the material. Patches made from this material include a plurality of apertures formed in the center portion. Another patent U.S. Pat. No. 6,755,781 relates to a sheet having one or more cuts disposed upon its length or apertures disposed through the full thickness of the sheet. A material used is chemically processed acellular human dermis which is freeze-dried for storage. U.S. Pat. No. 5,997,575 pertains to a submucosal tissue graft construct in the form of perforated laminate.

It can thus be seen that previous processes require extensive chemical treatment with a multitude of process steps in an attempt to obtain an acellular soft tissue specimen, which has limited shelf life.

### SUMMARY OF THE INVENTION

The present invention is directed toward a process for use in the preparation of acellular, (essentially lacking in living cells and/or non-living cells) soft-tissue implants that are derived from tissue products derived from the skin of human donors. The decellularized grafts produced typically provide long-term durability and function when used in clinical applications.

In certain embodiments, the present invention relates to a process for preparing soft tissue for implant in a human and removes cellular components from tissue taken from human donors while decontaminating the tissue. In a particular embodiment, the process comprises the following:

(1) obtaining donor skin from a human;

(2) removing hair from the recovered skin;

(3) processing and decellularizing the skin by soaking the tissue in sodium chloride and a detergent depending and rinsing same with sterile water to substantially remove the residual sodium chloride and detergent;

(4) disinfecting the skin by soaking the tissue in an antibiotic composition and rinsing same to remove residual chemicals to less than 1 ppm;

(5) processing the tissue by cutting the tissue to size; and

(6) packaging the tissue.

In certain embodiments, donor skin is obtained from a living donor, from whom the skin is recovered by surgery.

In a particular embodiment, the process comprises (1) obtaining donor skin from a human; (2) removing hair from the skin; (3) decellularizing the skin including inspection for visual defects; (4) soaking the tissue in a detergent and rinsing same with sterile water and disinfecting the skin in a disinfection solution containing about 0.5% to about 0.7% peracetic acid; and (5) processing the tissue by cutting the tissue to size.

It is thus an object of the invention to provide acellular allograft dermis for implantation into a human being.

It is another object of the invention to provide acellular disinfected allograft dermis which is packaged for usage as an implant by a surgeon.

It is still another object of the invention to provide acellular disinfected or decontaminated dermis which can be stored for long periods of time for later use by a surgeon for implantation into a human being.

In yet other embodiments, the present invention relates to a process for use in the preparation of acellular (essentially lacking in living cells and/or non-living cells) soft-tissue implants such as dermis, which have been lyophilized and must be rehydrated to obtain flexibility. The decellularized soft tissue implants produced typically provide long-term durability, flexibility, and ability to hold quantities of fluid and be easily used by the surgeon when used in clinical applications.

In certain embodiments, the present invention provides a process for treating soft tissue to increase the porosity of the tissue. The process comprises (1) obtaining acellular dermis; (2) freezing the dermis at 20° C. for about 15 minutes or for a time period sufficient to stabilize the temperature of the dermis not under vacuum; (3) freezing the dermis at a second lower temperature of −20° C. for about 80 to 120 minutes, for example, 100 minutes not under vacuum; (4) freezing the dermis at a third lower temperature until the dermis is frozen solid or at −40° C. for 60 minutes not under vacuum; (5) placing the acellular dermis under vacuum at a temperature of −40° C. for 30 minutes; (6) drying the acellular dermis under vacuum at −10° C. to +10° C., for example, −5° C. for 300 to 500 minutes, for example 400 minutes; and (7) drying the acellular dermis under vacuum at 25° C. to 35° C., for example 25° C. for 100 to 400 minutes, for example 240 minutes.

It is thus an object of the invention to provide acellular porous disinfected soft tissue for implantation into a human being.

It is yet another object of the invention to provide acellular dermis which can be quickly hydrated for use by a surgeon.

In certain embodiments, the present invention relates to a method for the treatment of donor soft tissue to prepare the same for implantation into a human comprising (a) decellularizing the donor soft tissue for a period of time sufficient to accomplish same; (b) treating the donor soft tissue with a detergent for a period of time ranging from 24 to 48 hours; (c) treating the donor soft tissue in a disinfection solution for a period of time to accomplish disinfection of the tissue; (d) rinsing the donor soft tissue so that residue from the disinfection solution is less than 1 ppm; (f) cutting the treated tissue to a specific size; and (g) packaging the acellular soft tissue in a sealed package. Examples of suitable detergents include Triton X-100 and 0.1% polyethylene glycol mono ether. In certain embodiments, the disinfection solution comprises peracetic acid, ethanol, propylene glycol and sterile water. In a particular embodiment, the peracetic acid is present in a range of about 0.1% to about 1.0%.

Optionally, a protease inhibitor can be added during decellularization of the donor soft tissue. Examples of suitable protease inhibitors include Aminoethylbenzenesulfonyl fluoride HCL (Serine Proteases), Aprotinin (broad spectrum, serine proteases), Protease Inhibitor E-64 (Cysteine Proteases), Leupeptin, Hemisulfate Cysteine Proteases and trypsin-like proteases, Pepstatin A (Aspartic Proteases). Marmistat (MMP2).

The donor soft tissue may be taken from a cadaver or a living donor and is typically frozen prior to decellularizing.

In certain embodiments, prior to decellularization, any hairs on the soft tissue are removed chemically.

In certain embodiments, the disinfection solution comprises peracetic acid (v/v) 0.05%-5.0%; propylene glycol (v/v) 20%-60%; ethanol (undenatured) (v/v) 10%-50% and sterile water 30% to 40%.

In one embodiment, the donor soft tissue is decellularized by placement in a solution of NaCl for a period of time ranging from 12 to 48 hours. In other embodiments, the donor soft tissue is placed in a weak acid solution for a period of time ranging from 12 to 48 hours. Examples of suitable acids include acetic acid, boric acid and citric acid. In other embodiments, the donor soft tissue is placed in a weak base solution for a period of time ranging from 2 to 24 hours. Examples of suitable bases include ammonium hydroxide and sodium carbonate.

In other embodiments, the instant invention relates to a method for the treatment of skin to prepare the same for implantation into a human comprising (a) thawing frozen donor skin tissue; (b) soaking the trimmed donor skin tissue in a decellularizing solution from 12 to 24 hours to remove cells from the skin and slough or loosen the epidermal layer from the dermis; (c) rinsing the decellular mixture from the skin with sterile water; (d) cutting the treated dermis tissue to a specific size; (e) immersing the cut dermis tissue in a detergent; (f) rinsing the detergent mixture from the dermis tissue with sterile water; (g) soaking the dermis tissue in a disinfecting solution for a period of time to accomplish disinfection of the tissue; (h) rinsing the disinfected dermis tissue a plurality of times to reduce the disinfecting residue to less than 1 ppm; and (i) packaging the acellular dermis tissue in a sealed package. Examples of suitable detergents for use in (e) include Triton X-100 and 0.1% polyethylene glycol mono ether. Optionally, a protease inhibitor may be added during decellularization.

In certain embodiments, the disinfection solution comprises peracetic acid, ethanol, propylene glycol and sterile water. In a particular embodiment, the peracetic acid is present in a range of about 0.5% to about 0.7%. In yet other embodiments, the disinfection solution comprises peracetic acid (v/v) 0.05%-5.0%; propylene glycol (v/v) 20%-60%; ethanol (undenatured) (v/v) 10%-50% and sterile water 30% to 40%. In further embodiments, the disinfection solution comprises peracetic acid (v/v) 0.5%-0.7%; propylene glycol (v/v) about 37.5%; ethanol (undenatured) (v/v) about 3% to about 26% and sterile water about 35%. In certain embodiments, disinfection solution has a pH ranging from 3.2-3.8.

In certain embodiments, the donor soft tissue is decellularized by placement in a solution of NaCl for a period of time ranging from 12 to 48 hours. In other embodiments, the donor soft tissue is placed in a wash and solution for a period of time ranging from 12 to 48 hours. In yet other embodiments, the donor soft tissue is decllularized by placement in a weak base solution for a period of time ranging from 2 to 24 hours.

In certain embodiments, the instant invention relates to a method for the treatment of skin tissue to prepare the same for implantation into a human comprising (a) thawing the frozen skin tissue; (b) trimming the skin tissue for processing; (c) soaking the skin tissue in 1M NaCl from 12 to 24 hours removing the epidermal layer from the skin leaving the dermis; (d) rinsing the NaCl soaked dermis with sterile water a plurality of times to remove the NaCl; (e) disinfecting the skin in a disinfection solution containing less than 1.0% peracetic acid for at least 4 hours; (f) rinsing the skin with sterile water a plurality of times to reduce the acid content to a residual level less than 1 ppm; (g) cutting the treated dermis to a specific configuration; and (h) packaging the cut dermis in a sealed package.

In certain embodiments, the disinfection solution comprises 36% to 3% propylene glycol, 23% to 26% ethanol, 35% sterile water and peracetic acid ranging from 0.5% to 0.7%.

In yet other embodiments, the invention relates to a method for the treatment of skin obtained from a living donor to prepare the same for implantation into a human comprising: (a) removal of hair from the skin; (b) decellularizing the skin to obtain soft tissue; (c) disinfecting the soft tissue in disinfectant solution; (d) rinsing the disinfection solution from the soft tissue to less than 1 ppm; and (e) cutting the treated soft tissue to a specific size.

Examples of surgeries from which skin may be obtained include abdominoplasty, lipectomy, panniculectomy, brachioplasty, thighplasty, and a circumferential.

Optionally, protease inhibitors may be added when decellularizing the skin. Examples of suitable protease inhibitors include Aminoethylbenzenesulfonyl fluoride HCL (Serine Proteases), Aprotinin (broad spectrum, serine proteases), Protease Inhibitor E-64 (Cysteine Proteases), Leupeptin, Hemisulfate Cysteine Proteases and trypsin-like proteases, Pepstatin A (Aspartic Proteases) and Marmistat (MMP2).

In other embodiments, the instant invention relates to a method for the treatment of skin obtained from living donor tissue to prepare the same for implantation into a human comprising (a) removal of hair from the skin by application of a chemical solution; (b) decellularizing the skin to obtain dermis tissue; (c) disinfecting the dermis tissue in disinfectant solution; (d) rinsing the disinfection solution from the dermis tissue to less than 1 ppm; and (e) cutting the treated dermis tissue to a specific size.

In yet other embodiments, the instant invention relates to a method for the treatment of skin obtained from living donor tissue to prepare the same for implantation into a human comprising (a) transporting the skin from the donor site in a disinfection and decellularizing solution; (b) removal of hair from the skin; (c) decellularizing the skin to obtain dermis tissue; (d) disinfecting the dermis tissue in disinfectant solution; (e) rinsing the disinfection solution from the dermis tissue to less than 1 ppm; and (f) cutting the treated dermis tissue to a specific size.

In some embodiments, the present invention relates to a method for the treatment of skin obtained from living donor tissue to prepare the same for implantation into a human comprising: (a) removal of hair from the skin by application of a chemical solution; (b) decellularizing the skin; (c) disinfecting the skin in disinfectant solution; (d) rinsing the disinfection solution from the tissue to less than 1 ppm; and (e) cutting the treated tissue to a specific size. Examples of suitable chemical solutions for use in (a) include water, mineral oil, calcium thioglycolate, calcium hydroxide, ceteareth-20, sodium hydroxide, camellia oleifera extract, sunflower seed oil, fragrance, chromium hydroxide green, and alkaline soap. In certain embodiments, decellurization comprises soaking the skin in a NaCl solution and a detergent solution, such as Triton X-100. In a particular embodiment, the disinfection solution is a mixture including (v/v 0.5% to 0.7%) peracetic acid 35%, propylene glycol, ethanol and sterile water.

In other embodiments, the present invention relates to a method for the treatment of skin obtained from a living donor to prepare the same for implantation into a human comprising (a) removal of hair from the skin by physical means; (b) decellularizing the skin; (c) disinfecting the skin in disinfectant solution; (d) rinsing the disinfection solution from the tissue to less than 1 ppm; and (e) cutting the treated tissue to a specific size. Examples of suitable physical means in (b) include hot wax, microdermabrasion, non-heating type laser hair removal in ultra short pulse (USP) range, hair inhibition. In certain embodiments, decellurization comprises soaking the skin in NaCl solution and a detergent solution, such as Triton X-100. In a particular embodiment, the disinfection solution is a mixture including (v/v 0.5% to 0.7%) peracetic acid at 35%, propylene glycol, ethanol and sterile water.

In yet other embodiments, the instant invention relates to a method for the treatment of soft tissue to provide porosity for the same for implantation into a human comprising (a) conditioning acellular soft tissue for about 15 minutes at 20° C. without vacuum; (b) freezing acellular soft tissue at about −20° C. for 2.5 to 3 hours without vacuum; (c) freezing acellular soft tissue at about −40° C. for about 1 hour without vacuum; (d) drying the frozen acellular soft tissue in a range of about ‘5° C. to about 25° C. for a period of time to reduce the residual moisture of the soft tissue and (e) packaging the soft tissue in a sealed package.

In certain embodiments, the instant invention relates to a method for the treatment of soft tissue to provide porosity for the same for implantation into a human comprising (a) freezing acellular soft tissue for less than 0.5 hour at about −20° C. without vacuum; (b) freezing acellular soft tissue at about −20° C. for about 2.5 to 3 hours without vacuum; (c) freezing acellular soft tissue at about −40° C. for about 1 hour without vacuum; (d) drying the frozen acellular soft tissue at about −5° C. for a period of time to reduce the residual moisture of the soft tissue and (e) packaging the soft tissue in a sealed package. In a particular embodiment, the soft tissue is dermis. In certain embodiments, the frozen acellular soft tissue is dried in (d) under vacuum at 600 mTorr. In certain embodiments, the drying in (d) runs from about 360 minutes to about 460 minutes. In a particular embodiment, the drying runs for a time period of about 15 minutes.

In some embodiments, the method further comprises freezing the frozen acellular soft tissue at about −40° C. under vacuum for about 30 minutes. In further embodiments, the method comprises further comprising drying the frozen acellular soft tissue above 0° C. for about 100 to about 400 minutes under vacuum to reduce the residual moisture content of the soft tissue to less than 6%. In certain embodiments, the acellular soft tissue after treatment has a pore size ranging from 2.0μ to 200μ.

In other embodiments, the present invention relates to a method for the treatment of soft tissue to increase the porosity of the soft tissue comprising (a) freezing the soft tissue at a series of reduced temperatures without the application of vacuum to create solid ice particles in the soft tissue having a size greater than 2.0μ within the soft tissue; and (b) drying the soft tissue under vacuum to reduce the residual moisture of the soft tissue to less than 6% leaving the tissue with a plurality of cavities greater than 2.0μ. In certain embodiments, the soft tissue is dermis.

In another embodiment, the method further comprises freezing the frozen acellular soft tissue at −40° C. under vacuum for about 30 minutes.

In certain embodiments, the soft tissue (e.g., dermis) after treatment has a pore cavity size ranging from 2.0μ to 200μ.

In another embodiment, the present invention relates to a method for the treatment of dermis to increase the porosity of the dermis comprising (a) freezing the dermis at a series of reduced temperatures without the application of vacuum to create ice particles in the dermis having a size greater than 2.0μ within the soft tissue; and (b) drying the dermis under vacuum to reduce the residual moisture of the dermis to less than 6% leaving the dermis with a plurality of cavities having a size substantially the same as the ice particles formed during the freezing in (a).

In some embodiments, the cavities range from 2.0μ to 200μ.

In another embodiment, the method further comprises freezing the frozen acellular soft tissue at about −40° C. under vacuum for about 30 minutes.

In yet other embodiments, the instant invention relates to a method for the treatment of dermis tissue to prepare the same for implantation into a human comprising (a) conditioning acellular dermis tissue for less than 0.5 hour at about 20° C. without vacuum; (b) freezing acellular dermis tissue at about −20° C. for 2.5 to 3 hours without vacuum; (c) freezing acellular dermis tissue at −40° C. for about 1 hour without vacuum; (d) freezing the frozen acellular dermis tissue at −40° C. under vacuum for about 30 minutes; (e) drying the frozen acellular dermis tissue for 5 to 10 hours at about −5° C. under vacuum; (f) drying the acellular dermis at 25° C. for a period of time to reduce the residual moisture of the soft tissue to less than 6%; and (g) packaging the dermis tissue in a sealed package.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 is a schematic flow chart showing a soft tissue process.

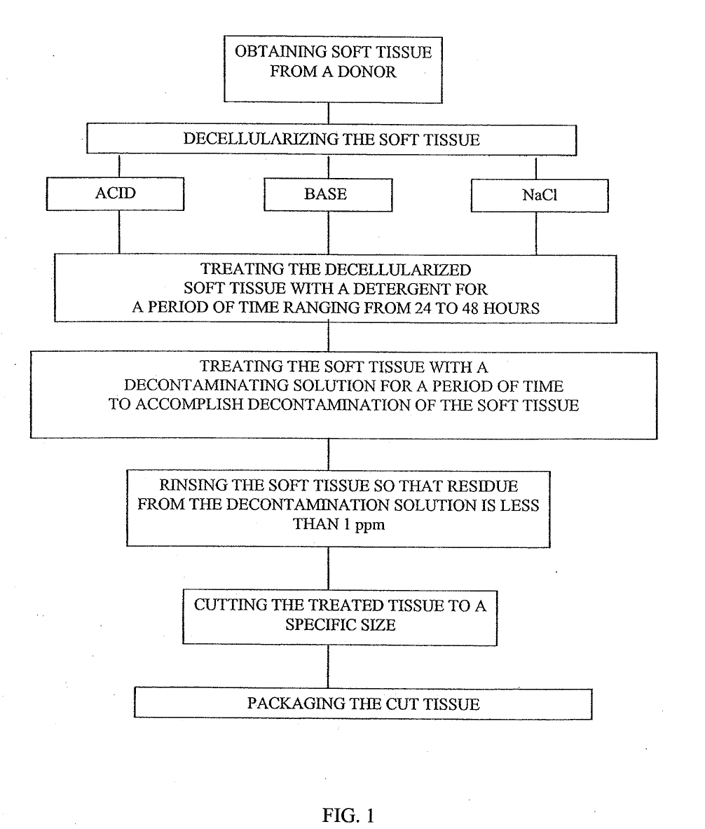

FIG. 2 is a schematic flow chart showing a soft tissue process, wherein the soft tissue is donor skin tissue from a living donor.

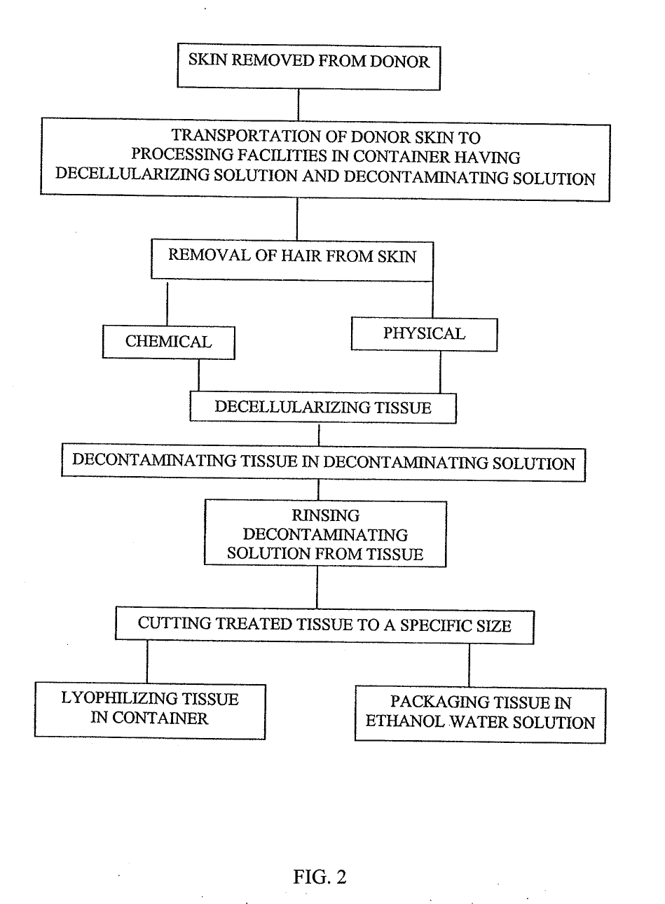

FIG. 3 is a schematic flow chart showing a treatment process for soft tissue.

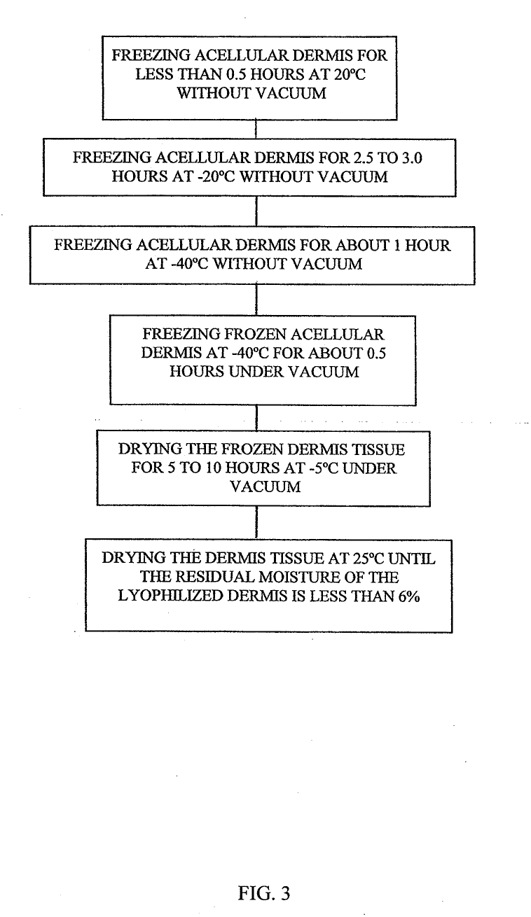

### DESCRIPTION OF THE INVENTION

In certain embodiments, the present invention relates to the preparation of skin from human donors, which is processed and decellularized. In certain further embodiments, the instant invention relates to a method for the preparation of skin from living donors, which is processed and decellularized. In other embodiments, the present invention provides a method for the preparation of soft tissue from human donors, which is decellularized and then treated to increase the tissue porosity.

For the purpose of this application, epidermis is the outer most layer of the skin and dermis is the layer of skin lying immediately under the epidermis and the term skin may refer to either epidermis, dermis or subcutaneous layers or all of the same, pending on the usage.

As used herein, the term “decontamination” refers to a process or treatment that renders a medical device, instrument, or environmental surface safe to handle. For example, according to OSHA, decontamination is “the use of physical or chemical means to remove, inactivate, or destroy bloodborne pathogens on a surface or item to the point where they are no longer capable of transmitting infectious particles and the surface or item is rendered safe for handling, use, or disposal” [29 CFR 1910.1030].

The term “disinfection” refers to the destruction of pathogenic and other kinds of microorganisms by physical or chemical means. Disinfection is generally less lethal than sterilization, because it destroys most recognized pathogenic microorganisms, but not necessarily all microbial forms, such as bacterial spores.

By “sterile” is meant completely free of all living microorganisms and viruses. The terms “sterilization” or “sterilized” refer to a process, after which the probability of a microorganism surviving on a surface or item subjected to the process is less than one in one million (10⁻⁶).

An “acellular soft tissue” is a tissue-derived biological matrix structure that is made from any of a wide range of soft tissues by removing all, or substantially all, viable cells and all detectable subcellular components and/or debris generated by cell death. As used herein, an acellular soft tissue lacking substantially all viable cells includes soft tissue in which the concentration of viable cells is less than about 1% (e.g., less than 0.1%, 0.01%, 0.001%, 0.0001%, 0.00001%, or 0.000001%) of that in the tissue or organ from which the acellular soft tissue was made. An acellular soft tissue may also include soft tissue comprising, after decellularization, about 25% or less of nucleic acid (e.g., DNA) that is present in normal cellularized soft tissue.

The methods and related compositions described herein relate to treating soft tissue, and in particular embodiments, decellularizing dermal tissue obtained from human donors. In other embodiments, the methods and related compositions described herein relate to treating soft tissue to increase its porosity. The novel methods described herein can be applied to any number of suitable tissue types, including dermis, fascia pericardia, dura, tendons, ligaments, and muscle.

Acellular soft tissue can be obtained from human sources, such as tissue from elective surgery or from a cadaver, or may be obtained from non-human sources, such as non-human primates (e.g., monkeys, baboon, chimpanzees), pigs, cows, horses, goats, sheep, dogs, cats, rabbits, guinea pigs, gerbils, hamsters, rats, or mice. In further embodiments, where the soft tissue is from a non-human source, the non-human source is a genetically engineered non-human animal, e.g., one that has been genetically engineered to lack an immunogenic epitope of collagen-containing material, such as a terminal α-galactose moiety.

In certain embodiments, the process uses allograft human skin which has been previously taken from a human donor. Typically, the soft tissue which is processed in embodiments of the invention is full thickness skin which includes epidermis, dermis and subcutaneous layers.

In certain embodiments, the process uses allograft human skin which has been previously taken from a living human donor undergoing surgery to remove excess skin and fat after excessive weight loss. Advantageously, the cells having been taken from a living human being are viable since the human donor is living as opposed to cadaveric skin sources. In certain embodiments, the soft tissue which is processed is full thickness skin which includes epidermis, dermis and subcutaneous layers. Suitable examples of skin for use in methods of the invention include skin that is obtained as the result of abdominoplasty, commonly known as “tummy tuck”, a cosmetic surgery procedure to reshape and firm the abdomen; lipectomy; panniculectomy; brachioplasty; thighplasty; and circumferential, an excision completely around a surface, such as a belt excision around the lower abdomen.

In further embodiments, the skin which has been previously obtained from the human donor is generally frozen after recovery. In embodiments where the skin is obtained from a living donor, the skin is typically shipped from the donor site in a pouch containing sterilization solution such as antibiotics, alcohol or mixtures of same mixed with a decellularizing solution such as Sodium Chloride and is then frozen. This minimizes contamination of the tissue and begins the epidermal separation from the dermal skin layer. Generally, the skin is placed in the pouch shortly after surgery.

The frozen skin is typically then taken from the freezer, the outer packaging (e.g., Kapak bag) is removed and thawed in a basin filled with sterile purified water. In certain embodiments, prior to processing, tissue is inspected for damage (holes or tears) and distinctive features (moles, warts, tattoos), which may be removed using a scalpel. Tissue is typically inspected for hair and the same may be removed using any one of a number of technique, including chemical removal using compositions such as (1) water, mineral oil, calcium thioglycolate, calcium hydroxide, ceteareth-20, sodium hydroxide, camellia oleifera extract, sunflower seed oil, fragrance, chromium hydroxide green and (2) alkaline soap and physical removal such as hot wax, hair inhibition, non-heating type laser hair removal in ultra short pulse (USP) range (at e.g., wavelength of 1552 nm and exposure time of 1.1 picosecond) and microdermabrasion. A visual inspection may be performed to ensure the skin tissue has uniform thickness. Thickness may be recorded using a thickness gauge. To identify the orientation (dermal or epidermal side) of tissue such as skin, the skin may be positioned such that, for example, the epidermis faces the processor and an incision may be cut into the upper left corner of each piece of tissue to indicate the epidermal side.

In embodiments where the soft tissue is dermal tissue, in processing the dermal tissue, the epidermal layer is removed and the dermis tissue is decellularized.

In processing, the epidermal layer is removed and the dermis is typically decellularized using, for example, Sodium Chloride (NaCl) solution at a concentration of 0.1-10M, for example, about 1M with a pH ranging from 5.0-9.0, for example, 6.8-7.2, and is agitated, for example, at a speed of 65 rpm on an orbital shaker for 1-96 hours, for example 12 hours to a maximum of 48 hours. After 12 hours, the container holding the skin is generally checked to ascertain if the epidermal layers have been sloughed off. If not, the container may be checked every 2 hours until the epidermis has sloughed off. The dermis is then typically removed and placed on, for example, a cutting board with the epidermal side up, and any remaining epidermal layer are picked off and discarded as well as any remaining hairs. In some embodiments, the remaining dermis pieces are replaced in the tissue flasks, filled with sterile water and agitated on the orbital shaker for about 15 minutes. In some embodiments, the sterile water is refreshed and the rinse procedure is repeated one more time for a total of two rinses.

In further embodiments, once the final rinse (e.g., sodium chloride soak or sterile water rinse) is complete, the dermis pieces may be trimmed into shaped pieces, for example, rectangular, by removing all of the rough edges of each piece with a scalpel. The trimmed dermis pieces are then typically immersed in a detergent such as Triton X-100 solution having a concentration of 0.01-10.0%, for example, about 0.1% with a pH ranging from 4.5-8.5, for example, 6.2-7.0 and agitated on the orbital shaker for 1-96 hours, for example, 24 hours to 48 hours. In further embodiments, the dermis is then placed in tissue flasks filled with sterile water, and agitated on the orbital shaker at 65 rpm for 15 minutes. The sterile water is typically refreshed and the rinse procedure is generally repeated a minimum of 7 more times for a total of 8 water rinses. In some embodiments, a residual detergent test may be performed on the rinsate after the 6^{th} water rinse to ensure the detergent has been adequately removed.

In certain embodiments, the acellular dermis is disinfected in a solution containing a disinfection solution of e.g., 35% peracetic acid, ethanol, e.g., 95% ethanol (undenatured), propylene glycol and sterile water. Without being bound to theory, the use of propylene glycol in the disinfection solution is thought to increase the porosity of the soft tissue being treated with the disinfection solution. It is believed, for example, that the propylene glycol increases the porosity of any collagen in the tissue. The disinfection mixture is generally stirred, e.g., with a magnetic stir bar for at least 15 minutes or until homogenous. The dermis is then typically soaked under vaccum and agitated at 65 rpm for 30 minutes to 12 hours, for example, 4 hours, at 4° C. to 40° C., for example, 20° C.-25° C. In particular embodiments, the disinfection solution is formed with peracetic acid (v/v) 0.05%-5.0%, for example, 0.5%-0.7%; propylene glycol (v/v) 20%-60%, for example, 37.5%; ethanol (undenatured) (v/v) 10%-50%, for example, 23% to 26% and sterile water 30% to 40%, for example, 35%. The disinfection solution typically has a pH ranging from 2.0-5.0, for example, 3.2-3.8. In further embodiments, the disinfected dermis is subjected to a rinse series with sterile water followed with agitation at 65 rpm under vacuum; two 5-minute rinses, followed by two 10-minute rinses, followed by two 15-minute rinses for a total of 6 rinses or until the peracetic acid has been adequately remove. In some embodiments, after the last rinse, a residual test may be performed on the rinsate to ensure that the peracetic acid has been adequately removed with less than 1 ppm remaining on the tissue.

In further embodiments, disinfected dermis tissue is cut to a final size. If desired, the dermis can be perforated with holes about 1.2 mm in diameter spaced from each other 2 to 3 mm formed by a punch process. In particular embodiments, the tissue is dipped in 70% ethanol (e.g., specially denatured alcohol such as SDA-3C, which is 100 parts 190 proof ethanol and 5 parts isopropyl alcohol by volume) and 30% water and packaged or treated to increase pore size and lyophilized.

Dermal tissue for use in surgery needs to be fully decellularized, e.g., endogenous cells normally present in and on the skin must be removed from the skin matrix. Skin tissue (dermis) is comprised of several types of protein; the most prominent protein present is type I collagen. Cells may be removed by causing the collagen structure to undergo reversible swelling. The swollen structure is then fully hydrated and slightly less dense. The spaces between the fibrils will increase and open up between the chemically intact but swollen collagen fibrils. The cells within and on the surface of the dermis can be lysed (swollen) and have the cell wall rupture. This facilitates the removal of the cells and cell fragments from the still swollen collagen matrix.

After washing the cell and cell fragments out of the swollen matrix, the collagen swelling can be reversed to return it to its native, intact collagen state with the original dermal microstructure.

This reversible swelling can be achieved by adjusting the pH of the collagen in the skin. Examples of methods that may be employed to accomplish this include: Treatment by either acidic or alkaline pH, washing of the skin to remove the cells and cell fragments and finally treating the skin with a buffer wash to return the skin to physiologic pH (7.4) and the attendant deswelling of the collagen in the dermis matrix.

In further embodiments, the disinfected dermis tissue is cut to a final size and the tissue is then placed in a freezing and lyophilization cycle, which is thought to increase porosity of the dermis tissue. In certain embodiments, the acellular dermis is conditioned (e.g., equilibrated) for 15 minutes at about 20° C. or for a time period sufficient to stabilize the temperature of the dermis with a temperature ramp up from ambient with no vacuum. In further embodiments, the acellular dermis is next frozen at about −20° C. for 80 minutes to 120 minutes, for example, 100 minutes with no vacuum and in a third step, the tissue is frozen at about −40° C. for 45 minutes to 75 minutes or until the dermis is frozen solid, for example, 1 hour (60 minutes) with no vacuum.

It is thought that this stepped freezing which is initially without vacuum causes small solid ice particles to form within the dermis creating cavities which make the dermis more flexible when hydrated for use in surgery or other medical operations. Under conventional lyophilization techniques, ice particles do not form in the size necessary to increase dermis porosity. In embodiments of the present invention, however, after a three step freezing treatment with the dermis not being under vacuum thereby allowing large ice particles to form within the dermis, the dermis is placed under vacuum, e.g., about 400 to about 800 m Ton, e.g., about 600 m Torr at −40° C. for an additional 30 minutes so that the soft tissue is placed under vacuum after which it is subjected to a drying phase at about −10° C. to +10° C., for example, −5° C. for 300 to 500 minutes, for example, 400 minutes under vacuum at, e.g., 600 mTorr. In further embodiments, the frozen lyophilized dermis is then dried, e.g., at 25° C. to 35° C., for example, 25° C. for 100 to 400 minutes, for example, 240 minutes under vacuum at, e.g., about 400 to about 800 mTorr, e.g., 600 mTorr until the residual moisture of the lyophilized dermis is less than 6% and the dermis has a porosity throughout its body formed by the cavities left by the removal of the solid frozen ice. In certain embodiments, the pore cavity size left by the water being removed from the ice particles formed in the dermis during the initial three freezing stages ranges from about 2.0μ to about 200μ and allows the dermis to soak up more saline or other moisturizing compound than if it was conventionally lyophilized and dried so that the same is flexible upon hydration in the operating room.

In further embodiments, the finished dermis implant is placed, e.g., in foil and a Tyvek® pouch. While the present description has used dermis as an example of soft tissue, it is to be understood that the applicable process applies to all soft tissue and not just dermis.

The following non-limiting examples further describe and enable one of ordinary skill in the art to make and use the present invention.

### Example 1

Dermis tissue is cut to a rectangular shape, the subcutaneous fat is removed and the tissue placed in a container with an aqueous solution of a weak acid, e.g., acetic acid. The aqueous acetic acid solution is at a concentration of 0.1 Molar and at pH 2.9. The tissue is immersed in the acetic acid solution for 2 to 24 hours, preferably12 hours and is agitated and maintained at room temperature (15° C. to 30° C.). After the acid wash period, the acid solution is discarded and the skin washed in isotonic saline for three rinses of twenty minutes each to remove the cells and cell fragments. The saline is discarded after each rinse. Finally the skin is rinsed in a neutral pH buffer, e.g., phosphate buffered saline at pH 7.4 until the skin stabilizes at pH 7.4 (measured by testing the skin with pH paper contacting the wet skin).

Other weak acids that may be used are 0.1 Normal boric acid at pH 5.2 or 0.1 N citric acid at pH 2.2.

### Example 2

Dermis tissue is cut to a rectangular shape, the subcutaneous fat is removed and the tissue placed in a container with an aqueous solution of a weak base, e.g., ammonium hydroxide. The ammonium hydroxide is at a concentration of 0.1 Molar and at a pH of 11.1. The tissue is immersed in the ammonium hydroxide solution for 12 hours (range 2 to 24 hours) and is agitated and maintained at room temperature (15° C. to 30° C.). After the alkaline wash period, the base solution is discarded and the skin washed in isotonic saline for three rinses of twenty minutes each. The saline is discarded after each rinse. Finally the skin is rinsed in a neutral pH buffer, e.g., phosphate buffered saline at pH 7.4 until the skin stabilizes at pH 7.4 (measured by testing the skin with pH paper contacting the wet skin).

Other weak bases that may be used are 0.1 Normal sodium bicarbonate at pH 8.4 or 0.1 N sodium carbonate at pH 11.6.

Another method of decellularizing the dermis tissue is shown in Example 3.

### Example 3

Sodium Chloride (NaCl) solution at a concentration of 0.1-10M, preferably about 1M with a pH ranging from 5.0-9.0, preferably 6.8-7.2, and is agitated at a speed of 65 rpm on an orbital shaker for 1-96 hours, preferably 12 hours to a maximum of 48 hours. After 12 hours, the container holding the skin is checked to ascertain if the epidermal layers have been sloughed off. If not, the container is inspected every 2 hours until the epidermis has sloughed off. The dermis is then removed and placed on a cutting surface with the epidermal side up and any remaining epidermal layer is removed and discarded as well as any remaining hairs. Optionally, the remaining dermis pieces are replaced in the tissue flasks, filled with sterile water and agitated on the orbital shaker for 15 minutes. The sterile water is refreshed and the rinse procedure is repeated one more time for a total of two rinses.

After decellularization is performed by Examples 1, 2 or 3, the final rinse is complete, the dermis pieces may be trimmed into shaped pieces, preferably rectangular, by removing all of the rough edges of each piece with a scalpel. The trimmed dermis pieces are then immersed in 0.1% polyethylene glycol mono ether or in 0.1% Triton X-100 solution having a concentration of 0.01-10.0%, preferably about 0.1% with a pH ranging from 4.5-8.5, preferably 6.2-7.0 and agitated on the orbital shaker for 1-96 hours, preferably 24 hours to 48 hours. The dermis is then placed in tissue flasks filled with sterile water, and agitated on the orbital shaker at 65 rpm for 15 minutes. The sterile water is refreshed and the rinse procedure is repeated a minimum of 7 more times for a total of 8 water rinses. Optionally, a residual detergent test is performed on the rinsate after the 6^{th} water rinse to ensure the detergent has been adequately removed.

The acellular dermis is subjected to disinfection in a solution containing peracetic acid, ethanol (undenatured), propylene glycol and sterile water. The disinfection mixture is stirred with magnetic stir bar for at least 15 minutes or until homogenous. Dermis tissue is soaked in the solution and agitated at 65 rpm for 30 minutes to 12 hours, preferably 4 hours, at 4° C. to 40° C. preferably 20° C.-25° C. The disinfection solution is formed with peracetic acid 35% (v/v) 0.05-5.0%, preferably 0.5-0.7%; propylene glycol (v/v) 20%-60%, preferably 37.5%; ethanol 95% (undenatured) (v/v) 10%-50%, preferably 23% to 26% and sterile water 30% to 40%, preferably 35%. The disinfection solution has a pH ranging from 2.0-5.0, preferably 3.2-3.8. The disinfected dermis is subjected to a rinse series with sterile water followed with agitation at 65 rpm under vacuum; two 5-minute rinses, followed by two 10-minute rinses, followed by two 15-minute rinses for a total of 6 rinses. Optionally, after the last rinse, the residual test is performed on the rinsate to ensure that the peracetic acid has been adequately removed with less than 1 ppm remaining on the tissue.

The disinfected dermis tissue is cut to finished size. If desired, the dermis can be perforated with holes about 1.2 mm in diameter spaced from each other 2 to 3 mm formed by a punch process. The tissue is dipped in 70% ethanol and 30% water and packaged or treated to increase pore size and lyophilized.

### Example 4

Treatment of Dermis Tissue Rinsing Decellurization shown in Example 3.

Frozen donor soft tissue is then thawed and then rinsed to maintain moisture. The thawed tissue is processed by removing hair and is then decellularized using 1M NaCl and 0.1% of Triton X-100. If desired at the time of decellularization one or more of the following protease inhibitors may be added; Aminoethylbenzenesulfonyl fluoride HCL (serine proteases) (25-100 Aprotinin (broad spectrum, serine proteases) (7.5-30 μm), Protease Inhibitor E-64 (cysteine proteases) (0.05-0.20 μm), Leupeptin, Hemisulfate (cysteine proteases) (0.05-0.20 μm), EDTA, Disodium (0.025-0.10 μm), and trypsin-like proteases, Pepstatin A (Aspartic Proteases). Marmistat (MMP2). The tissue is processed and decellularized and is inspected for visual defects and trimmed.

Once all blood and lipids are removed from the skin, the water is changed with clean sterile water. Defects (e.g., holes, tears, warts, tattoos) are removed from each piece of skin with a scalpel (epidermal side up during this process). Place each skin piece with the epidermal side up on the cutting board or flat surface, check the skin for damage (holes and initial tearing) and for distinctive features (mole, warts, tattoos) and cut these defects off using a scalpel.

Each piece is checked for hairs and the hairs are removed chemically by application of chemical compositions such as water, mineral oil, calcium thioglycolate, calcium hydroxide, ceteareth-20, sodium hydroxide, camellia oleifera extract, sunflower seed oil, fragrance, chromium hydroxide green after which the skin is rinsed with water. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge. The skin is decellularized in a sterile tissue culture bottle filled with 1 L of 1M NaCl. The bottle is sealed in a self-seal pouch and then placed the bottle on its flat side on the shaker with a set speed of 65 rpm for a period of 12-48 hours. The bottle(s) is checked after the first 12 hours to see if the epidermal layers have sloughed off. After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. The epidermal layers are removed with forceps and discarded leaving only the dermal layer (dermis). The bottles are rinsed with sterile water and the peeled skin pieces (dermis) are placed back into the bottle and filled with 1 L of 0.1% Triton X-100. (Alternatively, the bottles are then filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times. The bottle(s, are removed from the shaker, emptied and then filled with 1 L of 0.1% Triton X-100).

The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm's for irrigation to rinse off the Triton X-100. The rinse is repeated 7 more times for a total of 8 times. Optionally, after rinsing a residual detergent test is performed to make sure that the detergent has been removed from the tissue.

The dermis is soaked in disinfection solution for about 4 hours. The disinfection solution preferably is composed of peracetic acid, ethanol, propylene glycol and sterile water and the dermis is soaked and agitated at 65 rpm for about 4 hours at 20° C.-25° C. The disinfection solution contains peracetic acid (v/v), preferably 0.5%-0.7%; propylene glycol (v/v), preferably 36.0%-38.0%; ethanol (undenatured) (v/v), preferably 23%-26% and sterile water, preferably 35% -36%. The solution has a pH ranging from ranging from about 3.2-about 3.8. The canister stays on the shaker during the soak with the shaker set at 65 rpm. The dermis is initially rinsed in sterile water on the shaker at 65 rpm for 5 minutes and then rinsed 5 more times; 2^{nd} rinse for 5 minutes, 3^{rd} and 4^{th} rinse for 10 minutes and 5^{th} and 6^{th} rinse for 15 minutes. Optionally, after the 6th rinse, a test is performed for the presence of the peracetic acid. Less than 1 rpm must be present, otherwise additional rinses are required.

The strips of dermis are taken out of the canister using forceps and placed into a stainless steel basin. The basin is filled with water for irrigation and the residual detergent is rinsed from the surface of the skin. A wipe is placed on the top of a cutting board and moistened with sterile water. The skin is taken from the basin and laid on the cutting board epidermal side down (smooth side up) and measured.

After the 6^{th} rinse or upon later removal from the lyophilization, the dermis tissue is cut to size and may be perforated with the perforations 10 spaced 2-3 mm apart as shown in FIG. 4 with each perforation preferably having a diameter of about 1.2 mm.

The tissue may be lyophilized or may be immersed in 70% ethanol and 30% water and packaged for storage in sterile foil.

The tissue for lyophilization is laid flat on screens and placed in double Tyvek® pouches and each Tyvek® pouch is sealed. The package is stored flat in the freezer to prevent the tissue from becoming wrinkled or deformed until lyophilization.

The following examples show the treatment of dermis tissue using Sodium Chloride (NaCl) solution in the decellularization step but it is understood that the decellularization steps set forth in Examples 1 and 2 can be substituted for the Example 3 Sodium Chloride (NaCl) decellularization step.

### Example 5

Treatment of Dermis Tissue using the Decellularization Step Shown in Example 3

Frozen donor soft tissue is thawed and then rinsed to maintain moisture. The thawed tissue is processed and decellularized using 1M NaCl and 0.1% of Triton X-100. If desired at the time of decellularization one or more of the following protease inhibitors may be added; Aminoethylbenzenesulfonyl fluoride HCL (serine proteases) (25-100 μm, Aprotinin (broad spectrum, serine proteases) (7.5-30 μm), Protease Inhibitor E-64 (cysteine proteases) (0.05-0.20 μm), Leupeptin, Hemisulfate (cysteine proteases) (0.05-0.20 μm), EDTA, Disodium (0.025-0.10 μm), and trypsin-like proteases, Pepstatin A (Aspartic Proteases). Marmistat (MMP2). The tissue is processed and decellularized and is inspected for visual defects and trimmed.

Once all blood and lipids are removed from the skin, the water is changed with clean sterile water. Impurities are removed from each piece of skin with a scalpel (epidermal side up during this process). Place each skin piece with the epidermal side up on the cutting board or flat surface, check the skin for damage (holes and initial tearing) and for distinctive features (mole, warts, tattoos) and cut these defects off using a scalpel.

Each piece is checked for hairs and the hairs are removed physically by physical removals methods such as (1) hot wax, (2) hair inhibition, (3) non-heating type laser hair removal in ultra slow pulse (USP) range and (4) microdermabrasion. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge. The skin is decellularized in a sterile tissue culture bottle filled with 1 L of 1M NaCl. The bottle is sealed in a self-seal pouch and then placed the bottle on its flat side on the shaker with a set speed of 65 rpm for a period of 12-48 hours. The bottle(s) is checked after the first 12 hours to see if the epidermal layers have sloughed off. After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. The epidermal layers are peeled off with forceps and discarded leaving only the dermal layer (dermis).

Optionally, the bottles are rinsed with sterile water and the peeled skin pieces (dermis) are placed back into the bottle. The bottles are then filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times.

The bottle(s) are removed from the shaker, emptied, rinsed and filled with 1 L of 0.1% Triton X-100 and the dermis is replaced. The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm for irrigation to rinse off the Triton X-100. The rinse is repeated 7 more times for a total of 8 times. Optionally, after rinsing a residual detergent test is performed to make sure that the detergent has been removed from the tissue.

The dermis is soaked in disinfection solution for about 4 hours. The disinfection solution preferably is composed of peracetic acid, ethanol, propylene glycol and water and the dermis is soaked and agitated at 65 rpm for about 4 hours at 20° C.-25° C. The disinfection solution contains peracetic acid (v/v), preferably 0.5%-0.7%; propylene glycol (v/v), preferably 36.0%-38.0%; ethanol (undenatured) (v/v), preferably 23%-26% and sterile water, preferably 35%-36%. The solution has a pH ranging from ranging from about 3.2-about 3.8. The canister stays on the shaker during the soak with the shaker set at 65 rpm. The dermis is initially rinsed in sterile water on the shaker at 65 rpm for 5 minutes and then rinsed 5 more times; 2^{nd} rinse for 5 minutes, 3^{rd} and 4^{th} rinse for 10 minutes and 5^{th} and 6^{th} rinse for 15 minutes. Optionally, after the 6th rinse, a test is performed for the presence of the peracetic acid. Less than 1 rpm must be present, otherwise additional rinses are required.

The strips of dermis are taken out of the canister using forceps and placed into a stainless steel basin. The basin is filled with water for irrigation and the residual detergent is rinsed from the surface of the skin. A wipe is placed on the top of a cutting board and moistened with sterile water. The skin is taken from the basin and laid on the cutting board epidermal side down (smooth side up) and measured.

After the 6^{th} rinse or upon later removal from the lyophilization, the dermis tissue is cut to size and may be perforated with the perforations 10 spaced 2-3 mm apart as shown in FIG. 4 with each perforation preferably having a diameter of about 1.2 mm.

The tissue may be lyophilized or is immersed in 70% ethanol and 30% water and packaged for storage in sterile foil.

The tissue for lyophilization is laid flat on screens and placed in double Tyvek® pouches and each Tyvek® pouch is sealed. The package is stored flat in the freezer to prevent the tissue from becoming wrinkled or deformed until lyophilzation.

### Example 6

Treatment of Dermis from a Living Donor

Tissue which has been obtained from a living donor is shipped from the donor site in a pouch containing sterilization solution mixed with a decellularizing solution such as, for example, sodium chloride and then frozen.

Living donor tissue is then thawed and then rinsed to maintain moisture. The thawed tissue is processed by removing hair and is then decellularized using 1M NaCl and 0.1% of Triton X-100. If desired at the time of decellularization one or more of the following protease inhibitors may be added; Aminoethylbenzenesulfonyl fluoride HCL (serine proteases) (25-100 μm, Aprotinin (broad spectrum, serine proteases) (7.5-30:m), Protease Inhibitor E-64 (cysteine proteases) (0.05-0.20 μm), Leupeptin, Hemisulfate (cysteine proteases) (0.05-0.20 μm), EDTA, Disodium (0.025-0.10 μm), and trypsin-like proteases, Pepstatin A (Aspartic Proteases). Marmistat (MMP2). The tissue is processed and decellularized and is inspected visually for damage (e.g., holes or tears) and trimmed.

Once all blood and lipids are removed from the skin, the water is changed with clean sterile water. Defects (e.g., holes or tears) are removed from each piece of skin with a scalpel (epidermal side up during this process). Place each skin piece with the epidermal side up on the cutting board or flat surface, check the skin for damage (holes and initial tearing) and for distinctive features (mole, warts, tattoos) and cut these impurities off using a scalpel.

Each piece is checked for hairs and the hairs are removed chemically by application of chemical compositions such as water, mineral oil, calcium thioglycolate, calcium hydroxide, ceteareth-20, sodium hydroxide, camellia oleifera extract, sunflower seed oil, fragrance, chromium hydroxide green after which the skin is rinsed with water. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge. The skin is decellularized in a sterile tissue culture bottle filled with 1 L of 1M NaCl. The bottle is sealed in a self-seal pouch and then placed the bottle on its flat side on the shaker with a set speed of 65 rpm for a period of 12-48 hours. The bottle(s) is checked after the first 12 hours to see if the epidermal layers have sloughed off After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. Any remaining epidermal layers are removed with forceps and discarded leaving only the dermal layer (dermis). The bottles are rinsed with sterile water and the dermis is placed back into the bottle and filled with 1 L of 0.1% Triton X-100.

(Optionally, the bottles are instead filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times. The bottle(s),are removed from the shaker, emptied and then filled with 1 L of 0.1% Triton X-100).

The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm's for irrigation to rinse off the Triton X-100. The rinse is repeated 7 more times for a total of 8 times. Optionally, after rinsing a residual detergent test is performed to make sure that the detergent has been removed from the tissue so that less than 1 ppm is found on the tissue.

The dermis is soaked in disinfection solution for about 4 hours. The disinfection solution preferably is composed of peracetic acid, ethanol, propylene glycol and sterile water and the dermis is soaked and agitated at 65 rpm under vaccum for about 4 hours at 20° C.-25° C. The disinfection solution contains peracetic acid (v/v), preferably 0.5%-0.7%; propylene glycol (v/v), preferably 38.0%; ethanol (undenatured) (v/v), preferably 23%-26% and sterile water, preferably 35%-36%. The solution has a pH ranging from ranging from about 3.2-about 3.8. The canister stays on the shaker during the soak with the shaker set at 65 rpm. The dermis is initially rinsed in sterile water on the shaker at 65 rpm for 5 minutes and then rinsed 5 more times; 2^{nd} rinse for 5 minutes, 3^{rd} and 4^{th} rinse for 10 minutes and 5^{th} and 6^{th} rinse for 15 minutes. Optionally, after the 6th rinse, a test is performed for the presence of the peracetic acid. Less than 1 rpm must be present, otherwise additional rinses are required.

The strips of dermis are taken out of the canister using forceps and placed into a stainless steel basin. The basin is filled with water for irrigation to keep it moist. A wipe is placed on the top of a cutting surface and moistened with sterile water. The skin is taken from the basin and laid on the cutting surface epidermal side down (smooth side up) and inspected and measured.

If the dermis is to be lyophilized the skin is placed in a double Tyvek® pouch and the tissue placed in a freezer at −70° C. until lyophilization.

Optionally, after the 6^{th} rinse or upon later removal from the lyophilization, the dermis tissue is cut to size and may be perforated with the perforations 10 spaced 2-3 mm apart with each perforation preferably having a diameter of about 1.2 mm.

The tissue may be lyophilized or is immersed in 70% ethanol and 30% water and packaged for storage in sterile foil.

The tissue for lyophilization is laid flat on screens and placed in double Tyvek® pouches and each Tyvek® pouch is sealed. The package is stored flat in the freezer to prevent the tissue from becoming wrinkled or deformed.

### Example 7

Treatment of Dermis from a Living Donor

Tissue which has been obtained from a living donor is shipped from the donor site in a pouch containing, for example, disinfection solution mixed with a decellularizing solution such as sodium chloride and then frozen.

Living donor tissue is then thawed and then rinsed to maintain moisture. The thawed tissue is processed and decellularized using 1M NaCl and 0.1% of Triton X-100. If desired at the time of decellularization one or more of the following protease inhibitors may be added; Aminoethylbenzenesulfonyl fluoride HCL (serine proteases) (25-100 μm, Aprotinin (broad spectrum, serine proteases) (7.5-30 μm), Protease Inhibitor E-64 (cysteine proteases) (0.05-0.20 μm), Leupeptin, Hemisulfate (cysteine proteases) (0.05-0.20 μm), EDTA, Disodium (0.025-0.10 μm), and trypsin-like proteases, Pepstatin A (Aspartic Proteases). Marmistat (MMP2). The tissue is processed and decellularized and is inspected for visual defects and trimmed.

Once all blood and lipids are removed from the skin, the water is changed with clean sterile water. Defects (e.g., holes, tears, warts, tattoos) are removed from each piece of skin with e.g., a scalpel (epidermal side up during this process). Place each skin piece with the epidermal side up on the cutting board or flat surface, check the skin for damage (holes and initial tearing) and for distinctive features (mole, warts, tattoos) and cut these defects off using a scalpel.

Each piece is inspected for hairs and the hairs are removed physically by physical removals methods such as (1) hot wax, (2) hair inhibition, (3) non-heating type laser hair removal in ultra slow pulse (USP) range and (4) microdermabrasion. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge. The skin is decellularized in a sterile tissue culture bottle filled with 1 L of 1M NaCl. The bottle is sealed in a self-seal pouch and then placed the bottle on its flat side on the shaker with a set speed of 65 rpm for a period of 12-48 hours. The bottle(s) is checked after the first 12 hours to see if the epidermal layers have sloughed off. After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. The epidermal layers are removed with forceps and discarded leaving only the dermal layer (dermis). The bottles are rinsed with sterile water and the dermis is placed back into the bottle. The bottles are then filled with 1 L of 0.1% Triton X-100. Alternatively, the bottles are filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times. The bottle(s) are removed from the shaker, emptied and then filled with 1 L of 0.1% Triton X-100.

The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm's for irrigation to rinse off the Triton X-100. The rinse is repeated 7 more times for a total of 8 times. Optionally, after rinsing a residual detergent test is performed to make sure that the detergent has been removed from the tissue.

The dermis is soaked in disinfection solution for about 4 hours. The disinfection solution preferably is composed of peracetic acid, ethanol, propylene glycol and water and the dermis is soaked and agitated at 65 rpm under vacuum for about 4 hours at 20° C.-25° C. The disinfection solution contains peracetic acid 35% (v/v), preferably 0.5%-0.7%; propylene glycol (v/v), preferably 38.0%; ethanol 95% (undenatured) (v/v), preferably 23%-26% and sterile water, preferably 35% -36%. The solution has a pH ranging from ranging from about 3.2-about 3.8. The canister stays on the shaker during the soak with the shaker set at 65 rpm. The dermis is initially rinsed in sterile water on the shaker at 65 rpm for 5 minutes and then rinsed 5 more times; 2^{nd} rinse for 5 minutes, 3^{rd} and 4^{th} rinse for 10 minutes and 5^{th} and 6^{th} rinse for 15 minutes. Optionally, after the 6th rinse, a test is performed for the presence of the peracetic acid. Less than 1 rpm must be present, otherwise additional rinses are required.

The strips of dermis are taken out of the canister using forceps and placed into a stainless steel basin. The basin is filled with water for irrigation to keep it moist. A wipe is placed on the top of a cutting surface and moistened with sterile water. The skin is taken from the basin and laid on the cutting surface epidermal side down (smooth side up) and inspected and measured.

If the dermis is to be lyophilized the skin is placed in a double Tyvek® pouch and the tissue placed in a freezer at −70° C. until lyophilization.

Optionally, after the 6^{th} rinse or upon later removal from the lyophilization, the dermis tissue is cut to size and may be perforated with the perforations 10 spaced 2-3 mm apart with each perforation preferably having a diameter of about 1.2 mm.

The tissue may be lyophilized or is immersed in 70% ethanol and 30% water and packaged for storage in sterile foil.

The tissue for lyophilization is laid flat on screens and placed in double Tyvek® pouches and each Tyvek® pouch is sealed. The package is stored flat in the freezer to prevent the tissue from becoming wrinkled or deformed.

While specific embodiments of the subject invention have been discussed, the above specification is illustrative and not restrictive. One skilled in the art will appreciate that numerous changes and modifications can be made to the invention, and that such changes and modifications can be made without departing from the spirit and scope of the invention. The full scope of the invention should be determined by reference to the claims, along with their full scope of equivalents, and the specification, along with such variations.

Each patent, patent application, and publication cited or described in the present application is hereby incorporated by reference in its entirety as if each individual patent, patent application, or publication was specifically and individually indicated to be incorporated by reference.

1. A method for the treatment of donor soft tissue to prepare the same for implantation into a human comprising:
(a) decellularizing the donor soft tissue for a period of time sufficient to accomplish same;
(b) treating the donor soft tissue with a detergent for a period of time ranging from 24 to 48 hours;
(c) treating the donor soft tissue in a disinfection solution for a period of time to accomplish disinfection of the tissue;
(d) rinsing the donor soft tissue so that residue from the disinfection solution is less than 1 ppm;
(f) cutting the treated tissue to a specific size; and
(g) packaging the acellular soft tissue in a sealed package.

2. The method as claimed in claim 1 wherein said detergent is Triton X-100 or 0.1% polyethylene glycol mono ether.

3. (canceled)

4. The method as claimed in claim 1 wherein said disinfection solution comprises peracetic acid, ethanol, propylene glycol and sterile water.

5. (canceled)

6. The method as claimed in claim 1 wherein during (a), a protease inhibitor is added.

7. The method as claimed in claim 1 wherein said donor soft tissue is taken from a cadaver or a living donor and is frozen prior to decellularizing.

8-10. (canceled)

11. The method as claimed in claim 1 wherein in (a) the donor soft tissue is placed in a solution of for a period of time ranging from 12 to 48 hours wherein the solution is selected from the group consisting of: a solution of NaCl, a weak acid solution, and a weak base solution.

12-29. (canceled)

30. A method for the treatment of skin obtained from a living donor to prepare the same for implantation into a human comprising:
(a) removal of hair from the skin;
(b) decellularizing the skin to obtain soft tissue;
(c) disinfecting the soft tissue in disinfectant solution;
(d) rinsing the disinfection solution from the soft tissue to less than 1 ppm; and
(e) cutting the treated soft tissue to a specific size.

31. The method as claimed in claim 30 wherein said skin is obtained from an abdominoplasty, lipectomy, panniculectomy, brachioplasty, thighplasty, and circumferential.

32-36. (canceled)

37. The method as claimed in claim 30 wherein after (a), decellularizing adding one or more protease inhibitors selected from the group consisting of:
Aminoethylbenzenesulfonyl fluoride HCL (Serine Proteases), Aprotinin (broad spectrum, serine proteases), Protease Inhibitor E-64 (Cysteine Proteases), Leupeptin, Hemisulfate Cysteine Proteases and trypsin-like proteases, Pepstatin A (Aspartic Proteases). Marmistat (MMP2).

38-41. (canceled)

42. The method as claimed in claim 30 wherein the hair in (a) is removed from the skin by physical means or by application of a chemical solution.

43. The method as claimed in claim 30 wherein the decellurization comprises soaking the skin in a NaCl solution and a detergent solution.

44. (canceled)

45. The method as claimed in claim 30 wherein said disinfection solution is a mixture including (v/v 0.5% to 0.7%) peracetic acid 35%, propylene glycol, ethanol and sterile water.

46-52. (canceled)

53. The method as claimed in claim 43 wherein said detergent is Triton X-100.

54. (canceled)

55. A method for the treatment of soft tissue to provide porosity for the same for implantation into a human comprising:
(a) freezing acellular soft tissue at about −20° C. for 2.5 to 3 hours without vacuum;
(b) freezing acellular soft tissue at about −40° C. for about 1 hour without vacuum;
(c) drying the frozen acellular soft tissue in a range of about −5° C. to about 25° C. for a period of time to reduce the residual moisture of the soft tissue and
(d) packaging the soft tissue in a sealed package.

56. (canceled)

57. The method as claimed in claim 55 wherein said soft tissue is dermis.

58-60. (canceled)

61. The method as claimed in claim 55, further comprising freezing the frozen acellular soft tissue at about −40° C. under vacuum for about 30 minutes.

62. The method as claimed in claim 61, further comprising drying the frozen acellular soft tissue above 0° C. for about 100 to about 400 minutes under vacuum to reduce the residual moisture content of the soft tissue to less than 6%.

63-71. (canceled)

72. The method as claimed in claim 30 wherein said soft tissue is dermis.

73. The method of claim 55, wherein prior to (a), the acellular soft tissue is conditioned for about 15 minutes at 20° C. without vacuum.

74. The method of claim 55, wherein prior to (a), the acellular soft tissue is frozen for less than 0.5 hour at about −20° C. without vacuum.
